(19) United States
(12) Patent Application Publication
(10) Pub. No.: US 2022/0090155 A1
(43) Pub. Date: Mar. 24, 2022

(54) CHEMICALLY MODIFIED LIGNIN AS REDUCING AGENT FOR ENZYMATIC HYDROLYSIS OF LIGNOCELLULOSIC BIOMASS

(71) Applicant: Borregaard AS, Sarpsborg (NO)

(72) Inventors: Oskar Bengtsson, Sarpsborg (NO); Martin Lersch, Sarpsborg (NO); Anne Mari Kløften, Sarpsborg (NO)

(21) Appl. No.: 17/429,560

(22) PCT Filed: Feb. 14, 2020
(86) PCT No.: PCT/EP20/53865

§ 371 Date: Aug. 9, 2021

(30) Foreign Application Priority Data
Feb. 15, 2019 (EP) 19157510.9

(51) Int. Cl. C12P 19/14; D21C 11/02; D21C 5/00; D21C 3/06; D21C 11/00

(52) U.S. Cl. CPC C12P 19/14; D21C 11/02; D21C 11/0007; D21C 3/06; D21C 5/005

(57) ABSTRACT

The present invention relates to a method for increasing the rate of enzymatic hydrolysis of a polysaccharide substrate, said method comprising at least one step of: enzymatic hydrolysis of said substrate with a mixture of enzymes, said mixture comprising at least one enzyme selected from lytic polysaccharide monooxygenases; in the presence of chemically modified lignin, wherein during at least part of the time of said step of enzymatic hydrolysis, H₂O₂ is supplied to the reaction mixture comprising said substrate, said mixture of enzymes and said chemically modified lignin, either from an external source or by generation in situ.

### FIELD OF THE INVENTION

The present invention relates to a method for enzymatic hydrolysis of a polysaccharide substrate, said method comprising at least one step of: enzymatic hydrolysis of said substrate with a mixture of enzymes, said enzyme mixture comprising at least one enzyme selected from lytic polysaccharide monooxygenases; in the presence of chemically modified lignin, wherein during at least part of the time of said step of enzymatic hydrolysis, H₂O₂ is supplied to the reaction mixture comprising said substrate, said mixture of enzymes and said chemically modified lignin, either from an external source or by peroxide generation in situ.

### BACKGROUND

An increasing demand for energy, the depletion of fossil fuel resources and concern over global climate change has motivated an exploration of alternative feedstocks for fuels, chemicals and energy. Utilization of starch and other edible raw materials for the production of so called “first generation biofuels” is generally viewed as undesirable, because this use competes with the production of food and feed. Instead, non-edible lignocellulosic biomass is generally recognized as a potential sustainable resource to be utilized for the production of “second generation” fuels and chemicals. One example of such “second generation” feedstock is lignocellulosic biomass, which primarily comprises cellulose, hemicelluloses and lignin. The polysaccharides cellulose and hemicellulose may be enzymatically hydrolyzed into monosaccharides, which then may be converted into valuable chemicals.

According to one model, cellulose degradation of lignocellulosic biomass involves the synergistic action of at least three classes of enzymes. (1) endo-1,4-β-glucanases randomly cleave the internal bonds in the cellulose chains. (2) exo-1,4-β-glucanases attack the reducing or non-reducing ends of the cellulose polymer. Processive exo-1,4-β-glucanases are also referred to as “cellobiohydrolases” and are are among the most abundant components in naturally occurring and commercially available cellulase mixtures. (3) β-glucosidases convert cellobiose and short cellodextrins, the major products of the cellulose conversion based endo- and exo-glucanase mixture, into glucose. These three classes of enzymes are believed to act synergistically because endoglucanases generate new reducing and non-reducing chain ends for the exoglucanases. The released cellobiose is converted to glucose by β-glucosidases which relieves the glucanases of product inhibition. All these enzyme classes are “hydrolases”, i.e. cleave glycosidic bonds by addition of a water molecule.

The classical model for lignocellulose degradation has been modified more recently, based on the discovery of redox enzymes capable of cleaving glycosidic bonds. This novel class of enzymes, named “lytic polysaccharide monooxygenases” (LPMOs), encompasses enzymes that are copper-dependent metalloenzymes, active on polysaccharide substrates such as cellulose, hemicellulose, chitin and starch. Similar to endoglucanases, LPMOs introduce internal cleavages into the polysaccharide chains, creating new entry points for exo-acting enzymes. LPMOs are therefore believed to significantly enhance the activity of cellulolytic enzyme mixtures.

The reaction mechanism of LPMOs is still not (fully) known, although several different alternatives have been proposed. LPMOs are believed to be activated in the presence of molecular oxygen and an external source of electrons, i.e. a reducing agent, such as ascorbic acid (AscA). LPMOs are believed to not be able to enhance the hydrolysis of pure cellulose substrates, such as filter paper, Avicel and phosphoric-acid swollen cellulose in the absence of external reducing agents (see, for example: Harris, P. V et al., “Stimulation of Lignocellulosic Biomass Hydrolysis by Proteins of Glycoside Hydrolase Family 61: Structure and Function of a Large, Enigmatic Family”, Biochemistry 2010, 49, 3305-3316).

According to other prior art, it has been found that no external reducing agent is required to activate the LPMOs if water-insoluble lignin is present in the substrate. The synergistic effect of LPMOs was found to increase with the water-insoluble lignin content of the substrate [see, for example: Dimarogona, M. et al., “Lignin boosts the cellulase performance of a GH-61 enzyme from Sporotrichum thermophile” Bioresource Technology 2012, 110, 480-487 or Rodriguez-Zuniga et al., “Lignocellulose pretreatment technologies affect the level of enzymatic cellulose oxidation by LPMO”, Green Chemistry 2015, 17 (5), 2896-2903]. Therefore, in case lignocellulosic materials are used as the substrate for enzymatic hydrolysis, insoluble lignin may be seen as supplying the electrons needed for the oxidation step, thereby, in effect, acting as a reducing agent.

Several patent applications have been filed disclosing the addition of oxygen during enzymatic hydrolysis of lignocellulosic biomass (see, e.g., WO 2014/072393, WO 2015/035029 or WO 2016/029107). According to WO 2014/072393, oxygen should be added to the reaction in the form of gaseous bubbles, but only during a part of the reaction time. The preferred oxygen concentration during the aeration phase is also disclosed. The inventors propose the hypothesis that amorphous polysaccharides are hydrolyzed in the first part of the enzymatic hydrolysis and that the remaining crystalline polysaccharides are hydrolyzed in the second part. It is also disclosed that the addition of oxygen is especially beneficial during the hydrolysis of the crystalline polysaccharides. Similarly, according to WO 2015/035029 and WO 2016/029107, oxygen should be added during the saccharification of the cellulosic substrate and that the dissolved oxygen concentration should be maintained in this range during 25-75% of the saccharification period.

Different approaches to supply electrons and to increase LPMO activity have been disclosed. For example, it has been demonstrated that LPMO can be activated by light, in the presence of pigments (thylakoids or chlorophyllin) and an electron donor (AscA or insoluble lignin), see, for example: Cannella, D et al., “Light-driven oxidation of polysaccharides by photosynthetic pigments and a metalloenzyme”, Nature communications 2016, 7.

Recently, a different LPMO reaction mechanism was suggested, in which hydrogen peroxide (H₂O₂), instead of molecular oxygen, acts as the co-substrate[(see, for example, Bissaro, B.; Rohr, A. K.; Muller, G.; Chylenski, P.; Skaugen, M.; Forsberg, Z.; Horn, S. J.; Vaaje-Kolstad, G.; Eijsink, V. G. H., Oxidative cleavage of polysaccharides by monocopper enzymes depends on H2O2. Nat Chem Biol 2017, 13 (10), 1123-1128]. In this model, the LPMO reaction requires only one electron to prime the enzyme by reducing it from the LPMO-Cu(II) form to the LPMO-Cu(I) form. The activated LPMO then uses H₂O₂ for the hydrogen abstraction and subsequent hydroxylation of the substrate. A large LPMO activity increase on Avicel could be observed when H₂O₂ and AscA were supplemented to a reaction under anaerobic conditions, which supported this hypothesis. Stable reaction kinetics and high enzymatic rates were achieved by carefully controlling the H₂O₂ supply. However, rapid LPMO inactivation was observed under some conditions, such as when using the chlorophyllin/light-AscA system. The inactivation correlated with the H₂O₂ production potential of the system and was associated with accumulation of excess H₂O₂ in the reaction mixture. It was found that the inactivated LPMO had undergone several oxidative modifications that were mainly confined to the catalytic histidines [see, for example Bissaro, B.; Rohr, A. K.; Muller, G.; Chylenski, P.; Skaugen, M.; Forsberg, Z.; Horn, S. J.; Vaaje-Kolstad, G.; Eijsink, V. G. H., Oxidative cleavage of polysaccharides by monocopper enzymes depends on H2O2. Nat Chem Biol 2017, 13 (10), 1123-1128].

According to the prior art, increased rates of enzymatic hydrolysis of lignocellulosic biomass may be facilitated by the addition of hydrogen peroxide (H₂O₂), which activates lytic polysaccharide monooxygenase (LPMO) enzymes present in cellulase mixtures (see, for example WO 2018/060498). However, controlled addition of H₂O₂ to cellulosic biomass as the substrate and a mixture of enzymes is a challenge, in particular in a large scale process, and is, at any rate, associated with the risk of oxidative deactivation of the LPMOs.

Furthermore, supplementation of comparatively expensive reducing agents suggested in the art, such as AscA, leads to additional operating costs.

### SUMMARY OF THE INVENTION

Therefore, based on the prior art as discussed above, one objective of the present invention is to develop a more cost-effective method for enzymatic hydrolysis of polysaccharides, which minimizes or dispenses with the need to add costly reducing agents, and minimizes the risk of oxidative enzyme deactivation.

This objective and others, are solved by a method for enzymatic hydrolysis of a polysaccharide substrate, said method comprising at least one step of: enzymatic hydrolysis of said substrate with a mixture of enzymes, said mixture of enzymes comprising at least one enzyme selected from lytic polysaccharide monooxygenases; wherein said at least one step of enzymatic hydrolysis occurs in the presence of chemically modified lignin, wherein during at least part of the time of said step of enzymatic hydrolysis, H₂O₂ is added to said reaction mixture comprising at least said substrate, chemically modified lignin and said mixture of enzymes.

In embodiments of the present invention, the polysaccharide substrate comprises lignocellulosic biomass, preferably pretreated lignocellulosic biomass, preferably wherein the polysaccharide substrate consists of pretreated lignocellulosic biomass.

In embodiments of the present invention, H₂O₂ is added to said reaction mixture comprising said substrate, chemically modified lignin and said mixture of enzymes, either from an external source or by generation in situ.

In embodiments of the present invention the H₂O₂ is added indirectly by way of in-situ generation of H₂O₂, in particular by way of exposure of the reaction mixture to radiation, further particular UV or VIS radiation, or by enzymatic generation.

H₂O₂ may be added to the hydrolysis reaction at any suitable rate. In embodiments of the present invention, hydrogen peroxide is supplied to the reaction mixture at a rate of 10 to 5,000 μmoles hydrogen peroxide per liter reaction mixture per hour, preferably 20 to 1,000 μmoles hydrogen peroxide per liter reaction mixture per hour, further preferably 25 to 500 μmoles hydrogen peroxide per liter reaction mixture per hour.

It is desirable to add enough H₂O₂ to drive the LPMO activity and to maximize the overall hydrolysis yield. However, H₂O₂ accumulation should be avoided to keep the enzymes from being deactivated.

In embodiments of the present invention, the total amount of hydrogen peroxide supplied to the reaction mix is 2 to 1500 moles per ton of polysaccharide substrate, preferably 5 to 200 moles per ton substrate, more preferably 10 to 100 moles per ton substrate.

Other additives that enhance enzymatic hydrolysis, such as polyethylene glycol (PEG), may be added to the reaction mix.

In embodiments of the invention, the chemically modified lignin is water-soluble. In embodiments of the present invention, the chemically modified lignin comprises or consists of water-soluble lignin.

In embodiments of the present invention, the source for chemically modified lignin is spent sulfite liquor (SSL), i.e. the chemically modified lignin comprises spent sulfite liquor (SSL), preferably essentially consists of, spent sulfite liquor.

In accordance with the present invention, “essentially consisting of” means that at least 90% by weight relative to the overall weight chemically modified lignin (“w/w”) are spent sulfite liquor, preferably at least 95%, further preferably at least 99%.

In preferred embodiments, the source of said spent sulfite liquor is from a sulfite pretreatment step as used in the pulping of lignocellulosic biomass. Said sulfite pretreatment step converts at least a portion of insoluble lignin as present in lignocellulosic biomass into chemically modified water-soluble lignin.

Said chemically modified water-soluble lignin typically is present together with wood sugars, resins, organic acids and salts of sulfite and sulfate, which together make up the dry matter content of SSL.

In embodiments of the present invention, the dry matter content of SSL, in the mixture of the method of the present invention is 1.25 to 125 grams of dry weight chemically modified water-soluble lignin per liter of reaction mixture, preferably 2.5 to 65 grams of dry weight per liter, more preferably 6.5 to 32 grams of dry weight per liter.

Spent sulfite liquor (SSL) contains chemically modified water-soluble lignin that is believed to enhance the enzymatic hydrolysis of polysaccharides in the presence of LPMO enzymes and oxygen and/or H₂O₂.

Chemically modified lignin is believed to act as an external source of electrons, i.e. as a reducing agent, used to “activate” LPMO enzymes.

The cost of the chemically modified lignin is low compared to other reducing agents as known from the art as exemplarily discussed in the “Background” section. Utilization of chemically modified lignin as a reducing agent in enzymatic hydrolysis therefore reduces the costs of enzymatic hydrolysis, in particular in a large scale biorefinery.

Activation of LPMOs by addition of H₂O₂ in the presence of chemically modified lignin increases the efficiency of the hydrolysis process, which makes it possible to reduce the enzyme cost and/or the reactor size i.e. reduce capital expenditures.

Additionally, SSL is believed to provide a safe-guard against oxidative enzyme deactivation, at least based on the antioxidant properties of components contained in the SSL.

Therefore, an advantage of the method according to the present invention is that said method allows to minimize the enzyme loading in the hydrolysis process, while maintaining or even increasing the yield and productivity, thus improving cost-efficiency of the overall hydrolysis.

In embodiments of the present invention, spent sulfite liquor as formed during sulfite pulping (pretreatment) of cellulosic biomass enters the hydrolysis step together with at least part of the cellulosic substrate, in the form of residual SSL left in the lignocellulosic pulp after separating the liquid phase from the solid phase, or, optionally, after additional washing steps of the solid phase.

In the context of said sulfite pulping or sulfite pretreatment, the respective content of WO 2010/078930 is part of the present disclosure by way of incorporation by reference.

According to another embodiment, chemically modified lignin is added to the hydrolysis process from an external source. This may be advantageous in case the pretreatment process does not yield high enough concentrations of reducing agent.

In embodiments of the present invention, the amount of chemically modified lignin present in the reaction mixture comprising the polysaccharide substrate is from 1 to 100 grams of dry weight chemically modified lignin per liter of reaction mixture, preferably 2 to 50 grams of dry weight per liter, more preferably 5 to 25 grams of dry weight per liter.

In accordance with the present invention, the “reaction mixture” comprises all components that are present during the hydrolysis step. These components include said substrate, said enzyme mixture and said chemically modified lignin. This mixture also may contain other components, for example solvents or additives.

In embodiments of the present invention, the method for enzymatic hydrolysis according to any of the preceding embodiments, is a method in which the step of adding chemically modified lignin allows to lower the amount of enzymes vis-à-vis the same method not comprising said step of adding chemically modified lignin, wherein the two methods as compared are otherwise the same and lead to essentially the same C6 sugar yield. In preferred embodiments, the amount of enzymes is lowered by 5%, preferably by 10%.

In embodiments of the present invention, the method for enzymatic hydrolysis according to any of the preceding embodiments, is a method in which the step of adding chemically modified lignin allows to increase the C6 sugar yield vis-à-vis the same method not comprising said step of adding chemically modified lignin, wherein the two methods as compared are otherwise the same and use essentially the same amount and kind of enzymes, preferably wherein the C6 sugar yield is increased by 5%, preferably by 10%, preferably by 20%.

### BRIEF DESCRIPTION OF THE FIGURE

FIG. 1 shows the C6 sugar yields of enzymatic hydrolysis experiments Example 1, 2, 3 and 4.

### DETAILED DESCRIPTION OF THE INVENTION

### Feedstock (Substrate)

In accordance with the present invention, there is no specific restriction in regard to the kind or the origin of the polysaccharide substrate. In preferred embodiments the polysaccharide substrate is or comprises cellulose. In principle, the raw material for the cellulose may be any cellulosic material, in particular wood, annual plants, corn stover, corn cobs, cotton, flax, straw, ramie, bagasse (from sugar cane), suitable algae, jute, sugar beet, citrus fruits, waste from the food processing industry or energy crops or cellulose of bacterial origin or from animal origin, e.g. from tunicates, or polysaccharides from other marine sources.

In a preferred embodiment, wood-based materials are used as raw materials/substrate, either hardwood or softwood or both (in mixtures). Further preferably softwood is used as a raw material, either one kind or mixtures of different soft wood types. Bacterial microfibrillated cellulose is also preferred, due to its comparatively high purity.

In embodiments of the present invention, the feedstock is in its native state or it has been subjected to at least one pretreatment step to facilitate enzymatic hydrolysis (e.g. acid hydrolysis, steam explosion, ammonia fiber explosion (AFEX), alkaline wet oxidation, Kraft pulping, sulfite pulping).

In accordance with the present invention, any feedstock containing polysaccharides may be used since LPMOs per definition are oxidoreductases capable of cleaving glycosidic bonds present in polysaccharides. In embodiments of the present invention, enzymatic hydrolysis is performed, in particular, on cellulose, hemicellulose, starch and polysaccharides from marine sources such as chitin and fucoidan. Any polysaccharide substrate is suitable as long as LPMO enzymes are active on that substrate.

### Lignin

The chemically modified lignin preferably is a water-soluble lignin and can, for instance, be a sulfonated lignin, a carboxylated lignin, a hydrolysed carboxylated lignin, or an amine functionalized lignin.

In accordance with the present application, the term “lignin” relates to a biopolymer, respectively, a mixture of biopolymers, that is/are present in the support tissues of plants, in particular, in the cell walls providing rigidity to the plants. Lignin is a phenolic polymer, respectively, a mixture of a phenolic polymer. The composition of lignin depends on the plant and therefore varies depending on the plant it is derived from. Lignin in its native form, i.e., as present in the plant, is hydrophobic and aromatic.

In accordance with the present application, no restrictions exist in regards to the source of the lignin.

In accordance with the present application, the term “chemically modified lignin” is to be understood to relate to any lignin that is no longer present in its native form, but has been subjected to a chemical process. Processes for making chemically modified lignin are commonly known in the art.

The chemically modified lignin in accordance with the present invention is preferably water soluble and further preferably a sulfonated lignin. One preferred example of a sulfonated lignin is lignosulfonate. Lignosulfonate is obtained when lignin, respectively, lignin-containing cellulosic biomass, is subjected to sulfite cooking. Thus, lignosulfonate is the organic salt product recovered from digestion of wood (typically acid sulfite pulping with sulfurous acid). Preferred lignosulfonates can thus be described as water-soluble anionic polyelectrolyte polymers.

The term “lignosulfonate”, as used within the context of the present application, refers to any lignin derivative which is formed during sulfite pulping of lignin-containing material, such as, e.g., wood, in the presence of, for example, sulfur dioxide and sulfite ions, respectively, bisulfite ions.

For example, during the acidic sulfite pulping of lignin-based material, electrophilic carbon cations in the lignin are produced, which are a result of the acid catalyzed ether cleavage. Thus, lignin may react, via these carbo-cations, with the sulfite, respectively, bisulfite ions under the formation of lignosulfonate.

Another example of a chemically modified lignin is “Kraft lignin”. Kraft lignin is precipitated from Kraft alkaline pulping liquors, in particular from Kraft process pulp making during which the lignin has been broken down from its native form present in the wood pulp, representing molecular fractions of the original biopolymer. Kraft lignin can therefore be described as precipitated, unsulfonated alkaline lignin. Kraft lignin differs structurally and chemically from lignosulfonate, e.g., in that Kraft lignin is not watersoluble.

Kraft lignin can be further modified. The term “sulfonated lignin”, as used within the context of the present application, is to be understood as a lignin derivative in which sulfonic acid groups have been introduced. Thus, sulfonated lignin is characterized by the presence of —SO₃⁻M⁺ groups, wherein M is a cation balancing the anionic charge of the —SO₃⁻ moiety and which is selected from alkali metal cation, in particular from Li⁺, Na⁺, or K⁺, Ca⁺⁺, or Mg⁺⁺, or ammonium cation NH₄⁺, or mixtures thereof.

In one embodiment of the invention, the chemically modified lignin is sulfonated lignin obtained from Kraft lignin. In embodiments, sulfonated lignin may be obtained when Kraft lignin is treated with alkali sulfite and alkylaldehyde at elevated temperature and pressure.

In embodiments, sulfonated lignin is a lignosulfonate obtained by modifying a lignosulfonate, for instance, by subjecting it to ion exchange, preferably by reacting it with sodium sulfate.

### Sulfite Pretreatment

In a preferred embodiment, cellulosic biomass is used as a substrate in the present process, in particular lignocellulosic biomass, which does not require mechanical (pre)treatment, and wherein sulfite pretreatment (“cooking”) is the only (pre)treatment.

Sulfite cooking may be divided into four main groups: acid, acid bisulfite, weak alkaline and alkaline sulfite pulping.

In the preferred pretreatment in accordance with the present invention, the cellulosic biomass is cooked with a sulfite, preferably a sodium, calcium, ammonium or magnesium sulfite under acidic, neutral or basic conditions. This pretreatment step dissolves most of the lignin as sulfonated lignin (lignosulfonate; water-soluble lignin), together with parts of the hemicellulose.

The fact that lignocellulosic pulp resulting from this one-step pretreatment is particularly low in impurities, in particular lignin, makes it easier to develop or adapt enzymes for the hydrolysis.

Sulfite pretreatment is preferably performed according to one of the following embodiments. Therein and throughout the present disclosure, the “sulfite pretreatment” is also referred to as “cook”:
acidic cook (preferably SO₂ with a hydroxide, further preferably with Ca(OH)₂, NaOH, NH₄OH or Mg(OH)₂);
bisulfite cook (preferably SO₂ with a hydroxide, further preferably with NaOH, NH₄OH or Mg(OH)₂);
weak alkaline cook (preferably Na₂SO₃, further preferably with Na₂CO₃), and
alkaline cook (preferably Na₂SO₃ with a hydroxide, further preferably with NaOH).

In regard to the sulfite pretreatment step (sulfite cooking), which is a preferred pretreatment to be implemented prior to the enzymatic hydrolysis in accordance with the present invention, the respective disclosure of WO 2010/078930 with the title “Lignocellulosic Biomass Conversion” as filed on Dec. 16, 2009 is incorporated by reference into the present disclosure.

### Enzymatic Hydrolysis

In order to efficiently hydrolyze polysaccharides, it is important to maintain conditions that promote high total activity and ensure long term stability of the LPMO containing enzyme cocktail. Two relevant reaction parameters to consider are temperature and pH-value. Enzyme mixtures of fungal origin have an optimal performance at temperatures between 50-55° C. and within a pH interval of 5.0-5.5. However, other temperature and pH levels may be optimal, depending on the specific enzyme mixture used.

In order to ensure that temperature and pH are kept at their optimal levels, thorough mixing of the reaction mixture is preferred.

In accordance with the present invention, any LPMO containing enzyme mixture may be utilized. In preferred embodiments, in order to achieve synergy, enzyme mixtures contain endo-1,4-β-glucanases, exo-1,4-β-glucanases and β-glucosidases in optimized proportions. An enzyme loading sufficient to hydrolyze at least 70% of the substrate within 200 hours of reaction time is preferred.

The following summarizes the advantages that the method of the present invention is believed to have over the prior art.

The present invention provides a cost-effective method for enzymatic hydrolysis of polysaccharides in the presence of LPMO enzymes and H₂O₂, by:
providing a low cost reducing agent;
reducing the risk of oxidative enzyme deactivation.

LPMO enzymes enhance the activity of cellulolytic mixtures significantly by introducing cleavages into the polysaccharide chains, creating entry points for other enzymes. The other enzymes degrade the polysaccharides, exposing new surface areas for the LPMOs. This synergy makes it possible to minimize the enzyme loading in a enzymatic hydrolysis process, with maintained or even increased yield and productivity, thus improving the cost-efficiency of the process.

### EXAMPLES

### Example 1. Standard Enzymatic Hydrolysis of Spruce Pulp

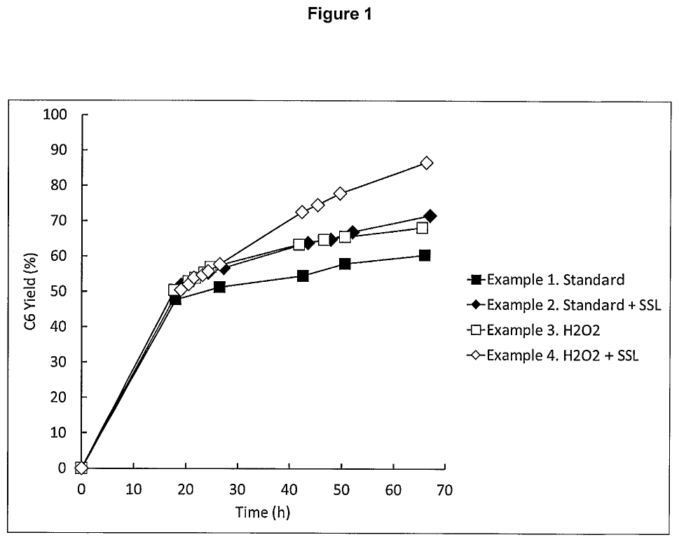

This experiment shows enzymatic hydrolysis of spruce pulp under standard conditions, (as described in Example 5). The C6 sugar yield was 60% after 66 hours of hydrolysis (see FIG. 1, lowermost curve). Hydrogen peroxide can be generated by LPMO enzymes from oxygen in the head space through an “empty” cycle, and a limited reducing capacity is provided by residual insoluble lignin as naturally present in the spruce pulp. However, the LPMO activity is restricted and the C6 sugar yield remains low throughout the reaction.

### Example 2. Standard Enzymatic Hydrolysis of Spruce Pulp with Added SSL

This experiment shows enzymatic hydrolysis of spruce pulp with 10 g/L of SSL dry matter added. The C6 sugar yield was 72% after 67 hours of hydrolysis (see FIG. 1, second curve from below). Hydrogen peroxide generated by LPMOs from head space oxygen together with the electrons provided by the reducing agent (chemically modified lignin) allows for increased LPMO activity, which is believed to explain the increased C6 sugar yield compared to Example 1.

### Example 3. Enzymatic Hydrolysis of Spruce Pulp with Addition of H₂O₂

This experiment shows enzymatic hydrolysis of spruce pulp, where 200 μmoles of hydrogen peroxide per liter reaction mixture per hour was continuously added after 20.5 hours of hydrolysis. The C6 sugar yield was 68% after 66 hours of hydrolysis (see FIG. 1, third curve from below). The C6 sugar yield increased compared to Example 1, which is explained by the availability of co-substrate (H₂O₂) and a limited reducing capacity provided by residual insoluble lignin in the spruce pulp, sufficient to yield some LPMO activity.

### Example 4. Enzymatic Hydrolysis of Spruce Pulp with Added SSL and H₂O₂ Addition

This example (in accordance with the present invention) shows LPMO enhanced enzymatic hydrolysis of spruce pulp with 10 g/L of SSL dry matter present, where 200 μmoles hydrogen peroxide per liter reaction mixture per hour was continuously added after 20.5 hours of hydrolysis. The C6 sugar yield was 87% after 66 hours of hydrolysis (see FIG. 1, uppermost curve). The C6 sugar yield increased significantly compared to Example 1, 2 and 3, which is believed to be due to the combined availability of co-substrate (H₂O₂) and reducing agent (chemically modified lignin).

### Materials and Methods

### Substrate, Additive and Enzymes

Sulfite-pulped Norway spruce (Picea abies) and SSL was obtained from a commercial scale sulfite pulp mill (Borregaard AS, Norway). Commercial cellulase mixture Cellic® CTec3 was obtained from Novozymes A/S, Denmark.

### Experimental Conditions

Enzymatic hydrolysis experiments were conducted in a 3.6 L bioreactor (Labfors 5 BioEtOH reactor, Infors-HT, Bottmingen, Switzerland) with 1.8 L working volume, a substrate loading of 12% (w/w) dry matter (DM) of sulfite-pulped Norway spruce and an enzyme loading of 4% (w enzyme/w substrate) of commercial cellulase cocktail Cellic® CTec3. The temperature was 50° C., pH 5 was maintained by automatic addition of 1 M NaOH and the stirring rate was 250 rpm. Hydrolysis reactions were started up as follows. All liquids, including SSL if applicable, but except enzymes, were added to the reactor together with approximately ⅓ of the wet pulp. The reactor was then heated to 50° C. and pH adjusted to approximately 5.1 using 7.5N NaOH. Once the pH and temperature were correct and stable, the enzyme mixture was added.

The reactor was then left for about 5 minutes to allow for the enzymes to blend in before the rest of the pulp was added. The reactor was then left over night for liquefaction. Automatic pH control was started after liquefaction. In case H₂O₂ was added, H₂O₂ feeding was started 20.5 hours after initiation of the reaction, after liquefaction, to ensure sufficient mixing. H₂O₂ was delivered continuously using a Masterflex L/S Standard Digital peristaltic pump (Cole-Parmer, Vernon Hills, USA); the H₂O₂ feed rate was 200 μmoles hydrogen peroxide per liter reaction mixture h⁻¹. SSL was added to a concentration of 10 g/L of dry matter, in experiments where SSL was added.

### Sample Preparation

1.5-2 ml samples were withdrawn at various times throughout the reaction. The samples were centrifuged and kept in the freezer until analysis. Samples for analysis of sulfite and sulfate were diluted in a formaldehyde-solution and kept in a refrigerator until analysis.

### Analysis of Sugars and Yield Calculations

The sugar monomers were analyzed using Agilent HPLC with RI detector, on a Bio-Rad Aminex HPX-87P cation exchange column using MQ-water as mobile phase. The samples were diluted with MilliQ-water and filtered before analysis. C6 sugar yields were calculated according to Zhu, Y. Et al., «Calculating sugar yields in high solids hydrolysis of biomass». Bioresource Technology 2011, 102, 2897-2903.

### List of Abbreviations Used

AscA Ascorbic acid
GMC Glucose-methanol-choline
H₂O₂ Hydrogen peroxide
LPMO Lytic polysaccharide monooxygenase
SSL Spent sulfite liquor
LS Lignosulfonate
PEG Polyethylene glycol
V—TiO₂ Vanadium-doped titanium dioxide

1. A method for enzymatic hydrolysis of a polysaccharide substrate, said method comprising at least one step of:
enzymatic hydrolysis of said substrate with a mixture of enzymes, said mixture of enzymes comprising at least one enzyme selected from lytic polysaccharide monooxygenases,
wherein said at least one step of enzymatic hydrolysis occurs in the presence of chemically modified lignin, wherein during at least part of the time of said step of enzymatic hydrolysis, H₂O₂ is added to a reaction mixture comprising said substrate, chemically modified lignin and said mixture of enzymes.

2. The method according to claim 1, wherein the polysaccharide substrate comprises lignocellulosic biomass.

3. The method according to claim 1, wherein H₂O₂ is added directly to said reaction mixture comprising said substrate, chemically modified lignin and said mixture of enzymes.

4. The method according to claim 3, wherein H₂O₂ is added to the reaction mixture at a rate of 10 to 5,000 μmoles hydrogen peroxide per liter reaction mixture per hour.

5. The method according to claim 1 wherein a total amount of hydrogen peroxide added to the reaction mixture is 2 to 1,500 moles per ton of polysaccharide substrate.

6. The method according to claim 1, wherein an amount of chemically modified lignin present in the reaction mixture comprising the polysaccharide substrate is from 1 to 100 grams of dry weight chemically modified lignin per liter of reaction mixture.

7. The method according to claim 1, wherein the chemically modified lignin comprises spent sulfite liquor.

8. The method according to claim 7, wherein the spent sulfite liquor is from a sulfite pretreatment step of cellulosic biomass.

9. The method according to claim 8, wherein the spent sulfite liquor as formed during sulfite pretreatment of cellulosic biomass enters the hydrolysis step together with at least part of a cellulosic substrate, in the form of residual spent sulfite liquor left in lignocellulosic pulp after separating a liquid phase from a solid phase.

10. The method according to claim 7, wherein dry matter content of the spent sulfur liquor is 1.25 to 125 grams of dry weight spent sulfur liquor per liter of reaction mixture.

11. The method according to claim 1, comprising adding the chemically modified lignin to form the reaction mixture, wherein the step of adding the chemically modified lignin allows to lower an amount of enzymes vis-à-vis the same method not comprising said step of adding the chemically modified lignin, wherein the two methods as compared are otherwise the same and lead to essentially a same C6 sugar yield.

12. The method according to claim 11, wherein the amount of enzymes is lowered by 5%.

13. The method according to claim 1, comprising adding the chemically modified lignin to form the reaction mixture, wherein the step of adding the chemically modified lignin allows to increase a C6 sugar yield vis-à-vis the same method not comprising said step of adding the chemically modified lignin, wherein the two methods as compared are otherwise the same and use essentially a same amount and kind of enzymes.

14. The method according to claim 13, wherein the C6 sugar yield is increased by 5%.

15. The method according to claim 4, wherein H₂O₂ is added to the reaction mixture at a rate of 20 to 1,000 μmoles hydrogen peroxide per liter reaction mixture per hour.

16. The method according to claim 4, wherein H₂O₂ is added to the reaction mixture at a rate of 25 to 500 μmoles hydrogen peroxide per liter reaction mixture per hour.

17. The method according to claim 7, wherein the chemically modified lignin essentially consists of spent sulfite liquor.
